(19) United States
(12) Patent Application Publication
(10) Pub. No.: US 2002/0161352 A1
(43) Pub. Date: Oct. 31, 2002

(54) Vaginal ring preparation and application

(76) Inventors: Chen Hai Lin, Shanghai (CN); Shao Hai Hao, Shanghai (CN); Chen Jian Xing, Shanghai (CN); Chen Liang Kang, Shanghai (CN)

Correspondence Address: Joseph L. Strabala, Esq, Law Offices of Joseph L. Strabala, Suite 1020, One Embarcadero Center, San Francisco, CA, 94111, US

(21) Appl. No.: 10/134,402

(22) Filed: Apr. 25, 2002

(30) Foreign Application Priority Data
Apr. 25, 2001 (CN) 01112712.0

(51) Int. Cl. A61M 31/00

(52) U.S. Cl. 604/515; 604/279

(57) ABSTRACT

A vaginal drug delivery device includes a tubular base of an inert rubber composition, a first layer having a thickness up to 3 mm composed of a mixture of a drug to delivered, at least one surfactant and at least one dispersing agent applied to said outer surface of the tubular base, and a second layer of silicone rubber having a thickness up to 1 mm encapsulating the first layer on the tubular base whereby said drug will diffuse through said second layer when the device is inserted into the vagina to treat the patient with the drug in the first layer.

### BACKGROUND

This invention of vaginal ring is a new method in terms of the preparation method and its application.

Mifepristone is an anti-progesterone at the receptor level. It has the functions of terminating early pregnancy, interfering implantation, inducing menstruation, and promoting the mature of uterine cervix. It can compete with progesterone in achieving the effect of anti-progesterone. It also has certain ability of combining with glucocorticoid receptors. According to clinical experiments, Mifepristone can compete to combine with the estrogen and androgen receptors, reducing the available receptors that be combined with estrogen and androgen in uterine myoma tissue. It is used widely in clinic for the treatment of uterine myoma and endometriosis. In the same time, it could be used for contraceptive purpose.

However, the biological effectiveness is low if it is used orally. In order to reach certain treatment effects, the oral dose has to be increased to 10-25 mg/day and used for several months or years. This drug can combine with glucocorticoid receptors. The patients feel fatigue, nausea, dizziness, vomiting and other gastrointestinal side effects. When it is used to treat uterine myoma and endometriosis, this drug has to be used for a long time. Its side effects make it hard for patients to do so and limit this medicine in reaching its full effects.

Danazole is a synthesized derivative of 17- -methyl testosterone. It has minor similar effect as testosterone. It can inhibit the function of ovary by inhibiting the secretion of luteinizing hormone by corpus leteum. It can be used for the treatment of endometriosis, uterine endocrine diseases. It can also be used to shrink the myoma. However, the doses needed for this drug are large, 400-800 mg per day, lasting for more than 6 months, which costs a lot of money. Also the side effects of masculine, including acne, hirsutism, tense voice and body weight gain, are very apparent. In the same time, the ovary function is inhibited and the level of estrogen is below normal level. The menopause symptoms, such as sweating, palpitation, anxiety, are unacceptable for women.

Progesterone can be used to treat the early signs of abortion, functional uterine bleeding, uterine endometriosis, uterine endometrium adenoma. It is useless if taken orally. It should be taken parentally, such as injection, which is not acceptable for patients who need to take this medicine for a long time.

Selective estrogen antagonist, such as Raloxifene, Tamoxifene and Nafoxidine, need large dose for a long time period when used to treat endometriosis.

In order to treat uterine myoma, endometriosis more effectively, people want to find some methods, which could be used more conveniently, with less side effects and more apparent treatment effects.

In 1970, the idea of vaginal ring was proposed by Mishell and was used in clinical trial. Later, E2-ring, Levonorgestetrel vaginal ring, was invented for contraceptive purpose, hormone-replacing therapy in treating gynecological diseases. However, the drugs released from this kind of vaginal ring were less than 150 μg per day. The technique was not ready to be used to produce the vaginal ring that could release large quantity of insoluble drugs constantly.

There are a lot of similar products in the world that have been developed or are being developing which could not be used to release the insoluble drugs in a large quantity constantly for a long time.

The main purpose of this invention is to provide a constant, stable drug releasing system, like this vaginal ring, that could release drugs in large quantity. Also it provide a new area that vaginal ring could be used.

This purpose is achieved by the following steps:

1. The insoluble drugs are distributed molecularly in the dispenser. The special shapes of some dispensers, such as cylinder, middle-empty, etc., increase the contacting areas between the drugs and releasing and the releasing media, which increase the solubility of the drugs.

2. When the drugs are distributed molecularly in the dispensers and mechanically mixed with silicone or high molecular substances, the solubility of the drugs is impacted because of the interstitial characteristics of these high molecular substances. When the surface active agents are added:

1) The surface-active agents are inflated as the temperature is increasing during this process and the mechanical space and canal structures are formed within the silicone rubber or high molecular materials. This makes the drugs become more soluble

2) The biological availability of the drugs is improved because of their increased solubility due to the surface-active agents.

This invention is achieved by using a vaginal ring, which contains the drugs part and the silicone rubber covering the drugs.

This is a vaginal ring, which contains medicines and a silicone rubber layer covering the medicines.

The medicines account for 5-7% of total weight. The physiologically acceptable surface active agents account for 0.5-20% of total weight. The remains are the physiologically acceptable dispensers. The thickness of the silicone layer, which covers medicines, is 0.02-1 mm.

The medicines that contained in this vaginal ring are Mifepristone, Danazole, Progesterone, one or several selective estrogen antagonists including Raloxifene, Tamoxifene and Nafoxidine.

The physiologically acceptable surface active agents are obtained from one or more mixture of Span 20-80, Brij 52-76, OP emulsifiers, PEG 400-20000, Pluronic-124, Pluronic-188, Sodium lauryl sulphate, Sodium teradecyl sulpahte, Trolamine.

The physically acceptable dispensers mentioned above are obtained from one or more mixture of Glycerin, Methylethylene glycol, PEG 400-20000, Succinic acid, Cholic acid, Deoxycholic acid, Hexadecyl alcohol, Octadecul alcohol, B Type cyclodextrin, R Type cyclodextrin and silicone rubber. If one of the surface-active agents is PGE or Brij, the distribution agents should be different from the surface-active agents.

The silicone rubbers are obtained from HTV, RTV-2, RTV-1 or LTV, Docorning Medical Silicone Rubber Silastic-382 made in USA, Docorning Q7 Medical Silicone Rubber series made in USA, Docorning Implantable MDX series silicone rubber made in USA, or other medical silicone rubber.

The silicone rubber covering the medicines are obtained from HTV (RT silicon rubber with molecular weight of 0.3-1 million), RTV-2 (double composition RT silicon rubber with molecular weight of 0.0074-0.11 million), RTV-1 (single composition RT silicon rubber with molecular weight of 0.0074-0.11 million) or LTV (RT silicon rubber with molecular weight of 400-20000), Docorning Medical Silicone Rubber Silastic-382 made in USA, Docorning Q7 Medical Silicone Rubber series made in USA, Docorning Implantable MDX series silicone rubber made in USA, or other medical silicone rubber.

The medicines are contained in the center of vaginal ring and are surrounded by internal tubes, which could be made from medical silicone rubber. The center of the vaginal ring is empty. The thickness of the silicone rubber covering drugs is 0.02-mm.

### SUMMARY OF THE INVENTION

A vaginal drug delivery device includes a tubular base of an inert rubber composition, a first layer having a thickness up to 3 mm composed of a mixture of a drug to delivered, at least one surfactant and at least one dispersing agent applied to said outer surface of the tubular base, and a second layer of silicone rubber having a thickness up to 1 mm encapsulating the first layer on the tubular base whereby said drug will diffuse through said second layer when the device is inserted into the vagina to treat the patient with the drug in the first layer.

A preferred design for the tubular base is a ring.

### DESCRIPTION OF THE DRAWINGS

FIG. 1 is the illustration picture of this vaginal ring.

FIG. 2a is the cross section of the vaginal ring in one application plan.

FIG. 2b is the cross section of the vaginal ring in another application plan.

FIG. 2c is the cross section of the vaginal ring in the third application plan.

The following are the detailed description of this invention using these pictures as illustrations.

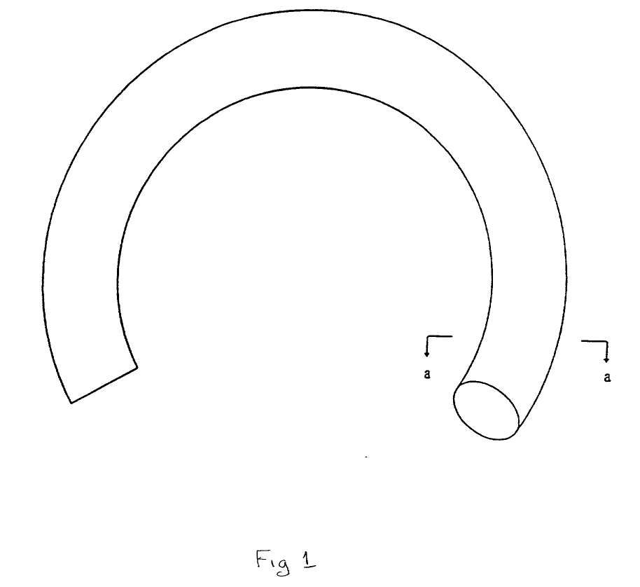

In FIG. 1, the diameter of the vaginal ring is 1-10 cm.

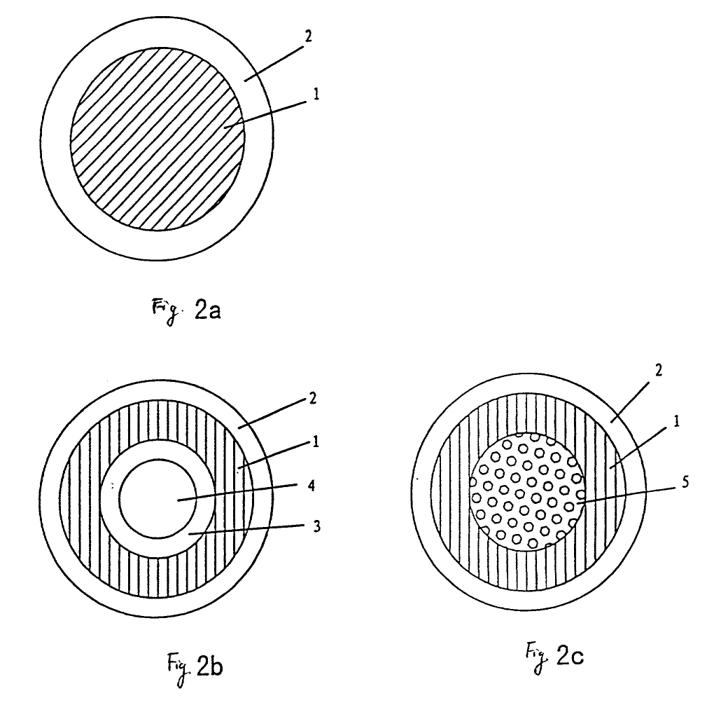

In FIG. 2a, part 1 (shadowed part) refers to the part containing medicines. Part 2 refers to the silicone rubber cover, which is 0.02-1 mm in thickness.

In FIG. 2b, part 1 (shadowed part) refers to the part containing medicines. Part 2 refers to the silicone rubber covering. Part 3 refers to the internal tubes, which are made of medical silicone rubber or other high molecular polymers. These internal tubes surround part 4, which is the empty core with diameter of 2.5-6.5 cm. The thickness of internal tube is 1-6 mm. The thickness of drugs is 0.5-3 mm. The thickness of the silicone rubber covering is 0.02-1 mm.

### DESCRIPTION OF A PREFERRED EMBODIMENT

The techniques used to produce this vaginal ring are some advanced techniques in this area, including injection molding process, mold molding process, squeezing out molding process, wetting process, etc. The following steps are included:

A. Prepare the medicine part based on the constitutions mentioned above. Then put the prepared drugs into the medical silicone rubber tubes and put the tubes in the modeling machine to form certain shape by heating or under pressure.

Or

B. (1) Prepare the silicone rubber tube to solid cylinder with the required size, which is the medical silicone rubber cylinder.

(2) Prepare the drugs according to the constitutions mentioned above and make them into thin layers.

(3) Cover the medical silicone rubber cylinder mentioned in (1) with the thin layers mentioned in (2).

(4) Cover the products got from (3) with another layer of medical silicone rubber with the thickness of 0.02-1 mm.

Or

C. (1) Choose the internal tubes that are made from medical silicone rubber or other chemical substances with diameter of 2.5-6.5 cm.

(2) Prepare the drugs based on the constitutions mentioned above and make them into thin layers.

(3) Cover the internal tubes with the thin layers we get from step (2).

(4) Cover another layer of medical silicone rubber with thickness of 0.02-1 mm.

Or

D. Put the silicone rubber into organic dissolvent, such as petroleum ether. Dip products got from B(3) or C(3) into this solution for 5 seconds and get them out to be dried.

This vaginal ring could be used to treat uterine myoma, uterine endometriosis and other related diseases of women. This product could also be used for contraceptive purpose. This vaginal ring could release drugs constantly with large quantity. The amount of drugs released could reach 1-10 mg per day. Also it has minor side effects.

The following are some further descriptions of this drug using some clinical applications:

Application 1:

Mixing 2.1 g progesterone, 0.1 g Sodium lauryl sulphate, 01 g Span-20, 0.7 g beta cyclodextrin (molecular weight is 1134, produced by Shanghai Testing Agents Company). Fill the mixture into the medical silicone rubber tube (silastic-382, thickness is 1 mm). Then model them into desire shape in the modeling machines by heating.

Application 2:

Using squeezing out molding process to make medical silicone rubber ring with 5 cm diameter with HTV medical silicone rubber.

Mixing 0.15 g Raloxifene, 0.015 g Brij 52 and 2.835 HTV medical silicone rubber (molecular weight is 0.3-1 million, produced by Shanghai Rubber Research Institute). Model the mixture into thin layers (thickness is 1 mm). Then cover the medical silicone rubber rings with these thin layers. Cover another layer of HTV medical silicone rubber (thickness is 0.02 mm) and model them into desired shape with the modeling machine.

Application 3:

Using squeezing out molding process to produce the medical silicone rubber internal tube (made from RTV-1, produced by Shanghai Rubber Institute) with diameter of 4 mm. Then make the silicone rubber tubes into shape of circle with diameter of 5 mm.

Mixing 0.3 g Mifepristone, 0.6 g Sodium lauryl sulphate, 0.3 Span-80 and 1.8 PEG 1200. Turn the mixture into thin layers. Then make thin layers with thickness of 0.02 mm from RTV-1 medical silicone rubber and cover the internal rubbers with these thin layers. Model the final product into desired shape under heating.

Application 4:

Put 1 g LTV (produced by Shanghai Rubber Research Institute) into 20 ml Petroleum ether and mix them together as dipping solution.

Model the HTV medical silicone rubber (molecular weight is 0.3-1 million, made by Shanghai Rubber Research Institute) into the shape of ring with diameter of 4 cm. Mix 1.5 Danazole, 0.03 Sodium lauryl sulphate and 1.47 PEG 20000 and make them into thin layers. Cover the medical silicone rubber rings with these thin layers and put the rings into the dipping solution we prepared above for 5 seconds. Then get them out to be dried.

Comparison Studies on these Application Cases:

Drug Releasing Test:

Devices Used:

1. High Pressure Liquid Chromatography (HPLC): Shimadzu LC-10AT

2. Shaking water bath: HZS-H

Process:

1. Tie the vaginal ring into a big test tube (125 ml). Add 100 ml distilled water into the tube under 37 C. Shake the testing tube for 24 hours with shaking rate of 60/minute. Then take the vaginal ring out.

2. The standard sample is the Mifepristone provided by Shanghai Family Planning Institute.

3. C-18-250 nm Analytic column.(Brand: Shimadzu): Pre-set HPLC. The testing conditions are:

Wavelength 310 nm.

Sensitivity: 0.1 AUFS

Speed: 3 Atten. 5

Flow phase: methanol:water=70:30 Moving speed=1 ml/minute

Injection amount: 10 ul Peak time: 5.48 minutes

Take 23 ul/ml standard sample, 10 ul injection sample to get the peak area of 656504--656800.

Prepare the calibration based on the amount contained in 100 ml.

Results:

The releasing amount in application 2 is: 1-2 mg/day

Clinical Test 1:

One female, 47 years old, had abnormal menstruations for the last two years. The amount of bleeding for each period was much more than normal and the period was irregular, with severe abdominal pain. She was diagnosed with uterine myoma in August 2000. In November 2000, it was found that the tumor enlarged a lot.

The patient began to use the vaginal ring mentioned in Application 3 on Nov. 13, 200. Her menstruation came back on Mar. 20, 2001, which means that this vaginal ring had been releasing effective amount of drugs constantly. She did not have abdominal pain and other discomfort feeling when the menstruation came back. The vaginal ring was removed on March 2001 and the uterus and myoma were found to be much smaller at that time.

Clinical Test 2:

A patient, 48 years old, was diagnosed with endometriosis and uterine myoma. She tried different kinds of treatment but could not achieve satisfied results. She had severe abdominal pain before and after menstruation and had to take analgesics to reduce the pain. In July 2000, she began to use the vaginal ring described in Application 3. After 10 days, pain disappeared. The menstruation came back in October 2000. A new ring was used after her menstruation. In January 2001, it was found that the myoma disappeared. Patient's self-feeling was quite good.

1. A vaginal drug delivery device comprising:
a tubular base of an inert rubber composition having an outer surface;
a first layer having a thickness from 0.3 mm to 3 mm applied to said outer surface of said tubular base, said first layer comprising a mixture of a drug to delivered, at least one surfactant and at least one dispersing agent; and
a second layer of silicone rubber having a thickness from 0.02 to 1 mm encapsulating said first layer on said tubular base whereby said drug will diffuse through said second layer when the device is inserted into the vagina to treat a patient with said drug.

2. The vaginal drug delivery device in claim 1 wherein the drug is selected from group consisting of Mifepristone®, Danazol®, Pegesterone ®, Ralozifene ®, Tamoxifen ® and Nafoxidine ®.

3. The vaginal drug delivery device in claim 1 wherein the surfactant is selected from one or more of the group consisting of Span 20-80, Brij 52-76, OP emulsifiers, Polyethylene glycol 400-20000, Pluronic-124, Pluronic-188, sodium lauryl sulphate, sodium teradecyl sulfate and Trolamine.

4. The vaginal drug delivery device in claim 1 wherein the dispersing agent selected from one or more of the agents from the group consisting of glycerin, methylethylene glycol, succinic acid, colic acid, deoxycholic acid, hexadecyl alcohol, octadecyl alcohol, B-type cyclodextrin, and R Type cyclodextrin.

5. The vaginal drug delivery device in claim 1 wherein mixture forming the first layer comprises by weight 5 to 7 percent of the drug, 0.5 to 20% surfactant and from 74.5 to 94.5 percent dispersing agent.

6. The vaginal drug delivery device in claim 1 wherein the silicone rubber is the second layer is selected from the group consisting of HTB, RTV-2, RTV-1, Docorning Medical Silicone Rubber Sitlastic-382, Docorning Medical Silicone Rubber Q7 and Docroning implantable MDX Silicone Rubber.

7. The vaginal drug device in claim 1 wherein the tubular base includes a hollow core.

8. The vaginal drug delivery device in claim 1 wherein the tubular base is a ring.

9. The vaginal drug delivery device in claim 1 wherein the tubular base is formed of silicone rubber selected from the group HTB, RTV-2, RTV-1, Docorning Medical Silicone Rubber Sitlastic-382, Docorning Medical Silicone Rubber Q7 and Docroning implantable MDX Silicone Rubber.

10. A method of vaginal delivery of drugs through gradual release comprising the steps of:
forming a inert base of a rubber composition;
applying a first layer on said inert base composed of a mixture of a drug to be administered, a surfactant and a dispersing agent, said first layer have a thickness less than 3 mm;
encapsulating said first layer with a second layer of silicone rubber, said second layer having a thickness of less than 1 mm; and
inserting said device into the vaginal cavity of a patient to be treated with the drug.
